(19) United States
(12) Patent Application Publication
(10) Pub. No.: US 2009/0162280 A1
(43) Pub. Date: Jun. 25, 2009

(54) DETECTING SOLUBLE A-BETA

(75) Inventors: Cristina Abucay Tan Hehir, Niskayuna, NY (US); Tiberiu Mircea Siclovan, Rexford, NY (US); Tunchiao Hubert Lam, Clifton Park, NY (US); Michael Christopher Montalto, Albany, NY (US); Richard Waltz Harper, Indianapolis, IN (US); Warren Jaye Porter, Indianapolis, IN (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: GENERAL ELECTRIC COMPANY, Schenectady, NY (US)

(21) Appl. No.: 11/961,460

(22) Filed: Dec. 20, 2007

(51) Int. Cl. A61K 51/04; G01N 33/53; A61K 49/00; C07C 229/00

(52) U.S. Cl. 424/1.65; 436/501; 435/7.1; 424/9.1; 562/452

(57) ABSTRACT

Agents, methods, and kits for qualitatively or quantitatively determining the presence of soluble A-beta in source of A-beta are provided, wherein the agents comprise the compound of formula III or the compound of formula III labeled with a signal generator.

### FIELD OF THE INVENTION

The present disclosure relates to agents useful for detecting soluble beta amyloid. The present disclosure also relates to methods for the detection of soluble A-beta.

### BACKGROUND

The main histopathological characteristic of Alzheimer's disease (“AD”) is the presence of neuritic plaques and tangles combined with associated inflammation in the brain. It is known that plaques are composed mainly of deposited, or insoluble in aqueous solution, fibrillar forms of the beta-amyloid (“A-beta”) peptide. The formation of fully fibrillar aggregated A-beta peptide is a complex process that is initiated by the cleavage of the amyloid precursor protein (“APP”). After cleavage of APP, the monomeric form of A-beta may associate with other monomers, presumably through hydrophobic interactions or domain swapping, to form dimers, trimers and higher-order oligomers. Oligomers of A-beta may further associate to form protofibrils and eventual fibrils, which is the main constituent of neuritic plaques. Soluble oligomers (soluble in aqueous buffer) of A-beta may contribute significantly to neuronal dysfunction. In fact, animal models suggest that lowering the amount of soluble A-beta peptide, without affecting the levels of A-beta in plaques, may improve cognitive function.

Presently, the definitive method of AD diagnosis is postmortem examination of brain for the presence of plaques and tangles. The antemortem diagnosis of AD is difficult, especially during the early stages, as AD symptoms are shared among a spectrum of other dementias. Currently, AD diagnosis is achieved using simple cognitive tests designed to test a patient's mental capacity such as, for example, the ADAS-cog (Alzheimer's disease assessment scale—cognitive subscale) or MMSE (Mini-mental state examination). The subjective nature and inherent patient variability is a major shortcoming of diagnosing AD by such cognitive means. The fact that AD cannot be accurately diagnosed early creates a formidable challenge for pharmaceutical companies that aim to test anti-A-beta drugs as therapy to slow or halt AD pathogenesis. Furthermore, even if AD could be detected early and patients could be treated with A-beta lowering compounds, there is currently no way to know if the therapy is clinically efficacious. A significant need exist for binders that bind to various species of A-beta for diagnostic and/or therapeutic applications. These binders may be used to measure soluble A-beta levels locally in the brain or they may be used as a research tool to investigate the properties of soluble A-beta in biological samples.

### SUMMARY

Provided herein are agents, methods, and kits for qualitatively or quantitatively determining the presence of soluble A-beta, wherein the agents comprise the compound of formula III or the compound of formula III labeled with a signal generator.

In some embodiments, the methods of detecting soluble A-beta, comprise providing a suspected or known source of A-beta; and applying the compound of formula III to the source of A-beta and observing the binding of the compound of formula III

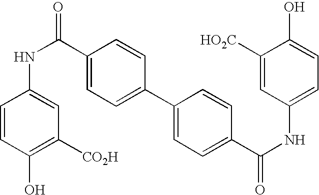

to the soluble A-beta present in the source of A-beta, and optionally quantifying the binding of compound of formula III to the A-beta present in the source.

In some embodiments, the methods further comprise the step of conjugating the compound of formula III with a signal generator selected from a radioisotope (e.g., 3H, 11C, 14C, 18F, 32P, 35S, 123I, 125I, 131I, 51Cr, 36Cl, 57Co, 59Fe, 75Se, and 152Eu), a paramagnetic particle (157Gd, 55Mn, 162Dy, 52Cr, and 56Fe), and an optical particle (e.g., a chromogenic or fluorescent dye) before applying the compound of formula III to the source of A-beta.

In another aspect, methods of determining the ability of a test agent to bind soluble A-beta are provided. The methods of determining the ability of a test agent to bind an A-beta comprises the steps of: (a) contacting a mammalian brain tissue sample with at least one A-beta species; (b) applying a test agent to the brain tissue sample; (c) determining whether the test agent binds to the A-beta species; (d) repeating steps (a)-(c) using compound of formula III in place of the test agent. In some embodiments, the method of determining the ability of a test agent to bind to soluble A-beta may further comprise the step of determining the relative binding of the test agent and the compound of formula III. In some embodiments, the method further comprises repeating each of steps (a)-(c) are repeated using a validated nonbinder. Each of steps (a)-(c) may be performed in parallel or in tandem on both the test agent and the compound of formula III.

The contacting step may include introducing the at least one A-beta species into an intact mammalian brain before the applying step. In some embodiments, the amount of the A-beta species applied to the brain tissue sample is a concentration of about 0.1 pM to about 0.1 nM.

In another aspect, provided herein are compositions comprising the compound of formula III

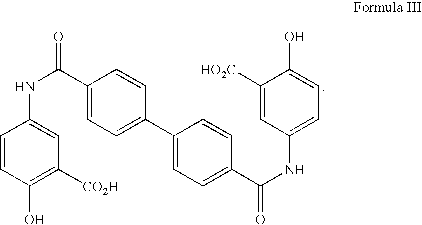

Also provided are compositions comprising the compound of formula III conjugated to a signal generator. A signal generator selected from a radioisotope, a paramagnetic particle, and an optical agent may be conjugated to the compound of formula III. In some embodiments, the signal generator comprises a radioisotope selected from 3H, 11C, 14C, 18F, 32P, 35S, 123I, 125I, 131I, 51 Cr, 36Cl, 57Co, 59Fe, 75Se, and 152Eu. In other embodiments, the signal generator comprises a paramagnetic particle selected from 157Gd, 55Mn, 162Dy, 52Cr, and 56Fe.

### FIGURES

These and other features, aspects, and advantages of the present invention will become better understood when the following detailed description is read with reference to the accompanying figures wherein:

FIG. 1 depicts SPA saturation binding curve of ³H-49b with soluble A-beta oligomers and fibrils on 49b.

FIG. 2 shows SPA self-competition assay between labeled and unlabeled 49b on soluble A-beta oligomers.

FIG. 3 compares binding of tritiated 49b with other tritiated probes in binding to soluble A-beta oligomers. Tritiated 49b was compared with non-related molecules such as tritiated cimetide, caffeine, and AZT.

FIG. 4 depicts SPA self-competition binding curve of ³H-PIB with A-beta fibrils.

FIG. 5 depicts SPA competition binding curve between ³H-PIB and unlabeled 49b on A-beta fibrils.

FIG. 6 depicts SPA competition binding curve between ³H-49b and unlabeled 353207 on soluble A-beta oligomers.

FIG. 7 depicts SPA competition binding curve between 3H-PIB and unlabeled compound of formula III (353207) on A-beta fibrils.

FIG. 8 depicts AFM images of soluble A-beta oligomers (Panel A) and fibrils (Panel B) used in the screening assay.

### DETAILED DESCRIPTION

The following detailed description is exemplary and not intended to limit the invention of the application and uses of the invention. Furthermore, there is no intention to be limited by any theory presented in the preceding background of the invention of the following detailed description of the drawings.

To more clearly and concisely describe and point out the subject matter of the claimed invention, the following definitions are provided for specific terms that are used in the following description and the claims appended hereto.

As used herein, the term “A-beta species” generally refers to the various forms of A-beta-derived polypeptide of 43 amino acid residues as follows: DAEFRHDSGYEVHHQKLVFFAEDVGSNKGAIIGLMVGGVVIAT (SEQ. ID. NO.: 1).

In general, A-beta may comprise amyloid polypeptides of varying length, various aggregation states, and/or solubility. The term “A-beta species” is intended to encompass A-beta species of varying polypeptide lengths. Thus, A-beta species may include various forms of A-beta amino acid residues 1 through 43 of the full-length A-beta peptide. Alternatively, the A-beta species may consist essentially of: residues 1-42 of the full length A-beta peptide, residues 1 through 40 of the full length A-beta peptide, residues 1-39 of the full length A-beta peptide, residues 1-38 of the full length A-beta peptide, residues 3-40 of the full length A-beta peptide, residues 3-42 of the full length A-beta peptide, residues 11-40 of the full length A-beta peptide, residues 11-42 of the full length A-beta peptide, residues 17-40 of the full length A-beta peptide, and residues 17-42 of the full length A-beta peptide.

The general term A-beta species also encompasses various forms of A-beta in several aggregation states (e.g., monomeric, soluble oligomers, or insoluble oligomeric). Because a variety of factors may affect which species of A-beta is found in solution, the aggregation state of the A-beta species may be selected according to the user's purposes by altering the A-beta polypeptide length, increasing or decreasing the concentration of A-beta present in a given aliquot of A-beta species, increasing or decreasing the temperature, pH, salt levels and metal content (e.g., Zn²⁺, Cu²⁺, etc.) of the given aliquot of A-beta species.

Various forms of soluble or insoluble A-beta species (regardless of the length of the polypeptide or the association state) may be derived from a variety of mammalian tissue sources, including but not limited to, brain tissue, cerebrospinal fluid, or blood serum. Alternatively, the A-beta species may be synthesized using art-recognized techniques such as protein expression systems or peptide synthesizers.

As used herein, the terms “fibrils” and “fibrillar” generally refer to A-beta preparations with largely beta-sheet content that are insoluble aggregates. Fibrils bind Congo Red and Thioflavin T dyes and cause these dyes to produce fluorescence signal. Fibril preparations preferentially comprise substantially fibrillar A-beta, but they may also comprise unsubstantial amounts of globular aggregates.

As used herein with regard to the A-beta species, the term “soluble” generally refers to nonaggregated A-beta peptides that are relatively stable and exhibit structural and functional characteristics that are distinct from the fibrillar amyloidogenic form of A-beta. In general the aggregation status of A-beta peptides may be broken into three categories: (1) micelles; (2) protofibrils; and (3) fibrils. The aggregation state of A-beta species may be determined using the techniques set out in Goldsbury et al., J Struct. Biol.; June; 130(2-3):352-62, (2000), in which samples are classified by the amount of β strands in undisturbed solution (pH 7.4 at 37° C.) by circular dichroism. Under these conditions (1) micelles demonstrate 0% β strands; (2) protofibrils demonstrate about 76% β strands; and (3) fibrils demonstrate 100% β strands. Soluble A-beta species for the assays of the invention contain only insubstantial amounts of protofibrils and fibrils.

As used herein, the term “signal generator” encompasses a substance that is capable of being detected by an imaging modality (e.g., optical detection or radiography). Examples of signal generators include, but are not limited to, fluorophores (e.g., cyanine dyes), radioisotopes, and paramagnetic ions.

As used herein, the term “specific binding” refers to the specific recognition of one of two different molecules for the other compared to substantially less recognition of other molecules. Thus, an agent that specifically binds a target molecule demonstrates affinities at least five-fold, and preferentially 10-fold to 100-fold affinities greater than non-binders.

As used herein, the term “species-specific binder” refers to any binder that preferentially attaches to one particular species of A-beta (e.g., soluble A-beta) relative to other species of A-beta (e.g., fibrillar A-beta).

Unless otherwise indicated, all numbers expressing quantities of ingredients, properties such as molecular weight, reaction conditions, so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

In one aspect the invention provides compounds and their derivatives that are useful as binders to soluble A-beta as represented by formula III:

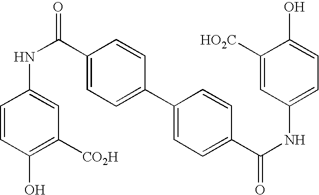

The compound of formula III that binds to A-beta species in a sample may be detected by the binder's emitted signal, such as emitted radiation, fluorescence emission, and optical properties of the agent. A signal generator may be conjugated to the compound of formula III and before. Alternatively, a signal generator such as a radioactive label may be attached to compound of formula III after compound of formula III binds to A-beta in a sample using, for example, sandwich assay methods.

### Detecting Soluble A-Beta

The present disclosure relates to methods of determining the presence and/or assessing levels of soluble A-beta in a sample or source that is either thought to or known to contain one or more A-beta species. The methods disclosed herein may be employed to diagnose or monitor amyloid-related diseases. In alternative embodiments, the disclosed methods may be used to qualitatively or quantitatively determine soluble A-beta levels from a given source.

In the detection methods, one or more A-beta species from a sample (e.g., tissue section) or a source (e.g., animal brain or CNS fluid) is contacted with the compound of formula III. The A-beta species may be derived from a variety of sources such as cell culture, post-mortem human tissue, animal models of disease, synthetic, and recombinant sources. In all embodiments, signal associated with binding may be converted to numerical values to quantify the amount of bound compound of formula III.

In all of the disclosed methods, the determining binding step may use any art recognized method of determine binding in a sample, for example optically observing the binding using fluorescence microscopy or radiographic imaging. Where the compound of formula III is conjugated with a moiety that has the ability to general a measurable signal when present in a sample (e.g. fluoresces or generates a radioactive signal), no additional agents are required for the determining binding step. For embodiments wherein the compound of formula III does not have the ability to generate signal, the disclosed methods may include additional steps of applying a signal generator to facilitate the binding determination step. Such an additional step may employ, for example, a labeled antibody that selectively adheres to the compound of formula III.

In one aspect, provided herein are methods for determining the ability of a test agent to bind an A-beta species comprising the steps of: (a) providing a source of A-beta; (b) contacting a test agent with a portion of the source of A-beta; (c) contacting an equivalent portion of the source of A-beta with the compound of formula III; (d) determining the ability of the test agent to bind to the A-beta present in the source relative to the ability of the compound of formula III to binding the A-beta. The contacting steps may occur in tandem or in parallel.

Additionally, compound of formula III may be used to determine that ability of a test agent to bind an A-beta species using competition assay methods. The competition assays methods comprise: (a) providing a source of A-beta; (b) contacting the source of A-beta with compound of formula III; (c) contacting a test agent with the source of A-beta; and (d) determining the ability of the test agent to bind to A-beta present in the source. In some embodiments, the competitive assays the amounts of compound of formula III and the test agent are substantially the same. In alternative embodiments, the amounts of compound of formula III and the test agent may be varied over a range of values and binding values normalized.

In some embodiments, the methods comprise: (a) contacting a mammalian brain tissue sample with at least one A-beta species; (b) applying a test agent to the brain tissue sample; and (c) determining whether the test agent binds specifically to the A-beta species; and (d) one or more control steps wherein each of steps (a)-(c) are repeated using a compound of formula III as validated binder in place of the test agent to provide a positive control. The additional control step (d) may be performed either in parallel or in tandem with the assay for the test agent. All embodiments may further include the additional step of determining the relative binding of the test agent and the compound of formula III. The disclosed methods may also include one or more washing steps following either or both the contacting and applying steps.

The step of applying an A-beta species to mammalian brain tissue may occur while the brain tissue is present in the animal or after the brain tissue has been removed from the animal. Accordingly, the contacting step may include introducing the at least one A-beta species into an intact mammalian brain before the applying step. In such embodiments, the A-beta species may introduced into a mammalian brain by any art recognized method (e.g., intracranial injection or intravascular injection). In alternative embodiments, the brain tissue is isolated from an intact brain prior to the applying step.

In embodiment where the source of A-beta is a brain tissue section, the brain tissue sample may be process according to standard pathology methods, for example a tissue section of about 10 microns to about 30 microns thick, applying a preserving agent to the brain tissue sample, and/or embedding the sample in a wax-type agent (e.g., paraffin).

The A-beta species applied to the brain tissue may be varied according the user's purposes and may substantially comprises a naturally occurring or synthetic monomers or multimers of A-beta (e.g., soluble or insoluble A-beta). Although, the amount of the A-beta species applied to the brain tissue sample may be varied, in some preferred embodiments the final a concentration of about 0.1 pM to about 0.1 nM.

In some embodiments, a signal generator is conjugated to the compound of formula III to facilitate the detection step. Examples of signal generators include, but are not limited to, fluorophores (e.g., cyanine dyes), radioisotopes, and paramagnetic ions. Suitable radioisotopes may include ³H, ¹¹C, ¹⁴C, ¹⁸F, ³²P, ³⁵S, ¹²³I, ¹²⁵I, ¹³¹I, ⁵¹Cr, ³⁶Cl, ⁵⁷Co, ⁵⁹Fe, ⁷⁵Se, and ¹⁵²Eu. Isotopes of halogens (such as chlorine, fluorine, bromine and iodine), and metals including technetium, yttrium, rhenium, and indium are also useful labels. Typical examples of metallic ions that may be used as signal generators include ^{99m}Tc, ¹²³I, ¹¹¹In, ¹³¹I, ⁹⁷Ru, ⁶⁷Cu, ⁶⁷Ga, ¹²⁵I, ⁶⁸Ga, ⁷²As, ⁸⁹Zr, and ²⁰¹Tl.

In yet another aspect, provided herein are kits for determining binding to A-beta comprising the compound of formula III. In some embodiments, the kit may further comprise one or a panel of A-beta species, which may be in solution or may be bound to a solid support (e.g., microtiter plate or a membrane). The kits may also include one or more signal generators bound to the compound of formula III or attached to a ligand for the compound of formula III, along with optional buffers for the various components of the kit.

### EXAMPLES

Practice of the invention will be still more fully understood from the following examples, which are presented herein for illustration only and should not be construed as limiting the invention in any way.

### Example 1

### A-Beta Species Formation

A. Soluble A-beta. 1 mg of human beta-amyloid 1-42 (H-1368, Bachem) and 500 uL of 1,1,1,3,3,3 hexafluoro-2-propanol (HFIP) (Aldrich) were chilled in separate bottles on ice for 30 minutes. Cold beta-amyloid 1-42 was solubilized with cold HFIP. The mixture was incubated for 1 hr at room temperature until it turned clear. The resulting solution was then dried to a film under vacuum. The film was dissolved again in cold HFIP and incubated another 1 hr at room temperature. The resulting solution was separated into aliquots in several microcentrifuge tubes. HFIP was removed under vacuum, and the films were stored at −20° C. until use. To prepare soluble oligomer, the film was dissolved in appropriate amount of dry DMSO (Sigma), and Ham's F12 media (Biosource) or PBS (Sigma, D8537) was added and incubated at 4° C. for 24 hours (final concentration of 200 uM or 0.9 mg/mL beta-amyloid in 2% DMSO). The tube containing the soluble oligomers was centrifuged at 13,000 rpm for 5 minutes, and its supernatant was transferred to a clean tube.

B. Insoluble A-beta (1-40) fibril formation: Lyophilized human beta-amyloid 1-40 peptide (Catalog number H-1194, Bachem) was solubilized with HFIP to produce a dry clear film following the process described above for beta-amyloid 1-42. The clear film of beta-amyloid 1-40 was diluted with 10 mM phosphate buffer, pH 7.4, containing 1 mM EDTA, such that final Aβ concentration was 0.5 mg/ml, then mixed briefly. The solution was incubated at 37° C. on an orbital shaker set at 200 rpm for 48 h. A cloudy solution was produced, and the fibril production was confirmed by measuring changes in fluorescence of Thioflavin T, as well as by atomic force microscopy and transmission electron microscopy. Fibrils were used immediately after their production was confirmed.

The three compounds shown in Table 1 were synthesized and evaluated for their ability to bind to soluble A-beta using the scintillation proximity assays (SPA) and/or AFM as described below. The percent inhibition reported was for 50 uM of the indicated unlabeled competitor and 130 nM of tritium labeled-49b.

TABLE 1

| For- | | | % |
|---|---|---|---|
| mu- | | | inhi- |
| la | | Structure | bition |
| | | No competitor | 0 |
| I | PIB | 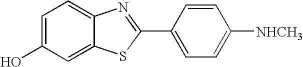 | 3 |
| II | 49b | 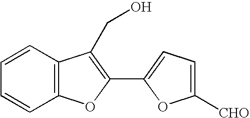 | 61 |
| III | 353207 | 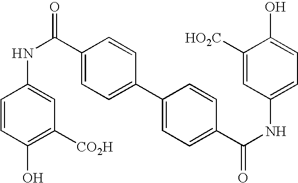 | 82 |

### Example 2

### Preparation of the Compound of Formula I (PIB)

PIB synthetic intermediates (steps 1-6 of Reaction scheme 1) were obtained according to published literature (Lowik, D. W. P. M., Tisi, L. C., Murray, J. A. H., Lowe, C. R. Synthesis, 2001, 12, 1780; McCapra, F., Razavi, Z. J. Chem. Soc., Chem. Commun. 1976, 5, 153). A new method was developed for the hydrolysis of 4-(6-(ethoxymethoxy)benzothiazol-2-yl)-N-methylaniline to PIB (2-(4-methylamino)phenylbenzothiazol-6-ol) and the final purification of PIB by reverse phase chromatography (steps 7-8 of Reaction scheme 1).

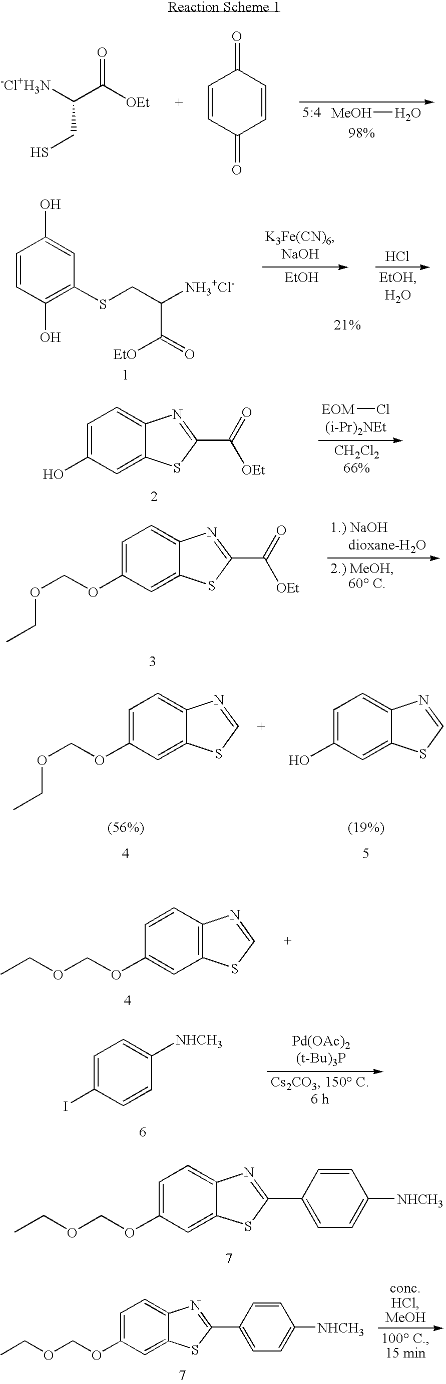

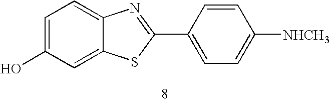

Under strictly anoxic conditions (glovebox), a solution of (t-Bu)₃P in dimethylacetamide (DMAC) was prepared in a custom made Schlenk flask from the original 1.0 g phosphine (90% technical) ampoule and 2 mL rigorously degassed DMAC. The approximate concentration of this stock solution was 1.48M. To a glass vial was added the benzothiazole 4 (83.3 mg, 0.38 mmol) (1.2 eq. vs. iodoaniline 6), iodoaniline 6 (221 mg, 0.95 mmol), Pd(OAc)2 (0.05 eq., 6 mg), and Cs₂CO₃ (272 mg, 2.20 eq). Degassed DMAC (10 mL/mmol of iodoaniline 6) was then added. The vial was capped with a fresh septum and the mixture was degassed by gently sparging with N₂ for 1 h. The phosphine solution was then added (0.1 eq. vs. iodoaniline 6, 2 eq. vs. Pd acetate) and the mixture was heated to 150° C. for 6 h. The solution was concentrated under reduced pressure (<50° C.), the crude product was adsorbed on silica gel, and purified by MPLC using hexanes/ethyl acetate gradient elution.

The hydrolysis of 4-(6-(ethoxymethoxy)benzothiazol-2-yl)-N-methylaniline to 2-(4-methylamino)phenylbenzothiazol-6-ol (PIB) and the final purification was performed by reverse phase HPLC. Concentrated HCl (100 μL) and MeOH (5 mL) was added to an 8 mL vial containing 4-(6-(ethoxymethoxy)benzothiazol-2-yl)-N-methylaniline (40 mg, 0.127 mmol). After heating for 15 min at 100° C., GCMS analysis of an aliquot (50 μL) showed complete conversion of the starting material. The resulting dark green reaction solution was then cooled down and the volatiles were removed by blowing it down to dryness. The residue was then purified by reverse phase HPLC using a C4 column. The fractions were combined and then lyophilized. PIB was obtained as a yellow green solid (15 mg, 46% yield). Purity analysis was confirmed by LCMS and was found to be consistent with previous report (Mathis, C. A., Wang, Y, Holt, D. P., Huang, G. F., Debnath, M. L. Klunk, W. E., J. Med. Chem., 2003, 46, 2740-2755).

### Example 3

### Preparation of the Compound of Formula II (49b)

A. Preparation of 2-bromo-3-bromomethyl benzofuran: 2-bromo-3-bromomethyl benzofuran was prepared as described previously, using a modified procedure (Helv. Chim. Acta 1947, 30, 297). To a solution of 3-methylbenzofuran (4 g, 30.26 mmol) in carbon tetrachloride (20 ml) was added benzoyl peroxide (100 mg) and recrystallized N-bromosuccinimide (NBS) (10.8 g, 2 equivalents). The mixture was refluxed for 3 hrs. The product formation was followed by GC-MS. Following analysis, 1.1 g NBS was added and the mixture was refluxed for 1 hr. At this point, one more addition of NBS (1 g) followed by 1 hr of reflux proved necessary. The solvent was stripped and replaced with ethanol (12 ml), the mixture was cooled to ⁻20° C., yielding a mass of yellow crystals, which were filtered at ⁻25° C. The crystals of 2-bromo-3-bromomethyl benzofuran were washed with ethanol 912 ml) at ⁻40° C., filtered and dried overnight (yield 7.672 g, 87%), better than 95% pure by GC-MS, which was immediately used in the next step. MS (m/e): 291, 290, 289 (M⁺), 211, 209, 183, 181, 146, 102, 75.

B. Preparation 2-bromo-3-hydroxymethyl benzofuran. 2-bromo-3-bromomethyl benzofuran from Step A (7.672 g, 26.45 mmol) was dissolved in dioxane (30 ml), followed by a solution of NaHCO₃ (2.67 g, 1.2 eq.) in water (30 ml). The mixture was refluxed for 1 hour while stirring vigorously, cooled to room temperature, diluted with water (150 ml) and extracted with dichloromethane (5×). The extract was washed with brine, dried over sodium sulfate, and the solvent was removed under reduced pressure. The resulting orange oil was dissolved in chloroform 912 ml and left to stand at ⁻20° C. The resulting yellow prisms were filtered at ⁻40° C., washed with chloroform and filtered cold. Yield: 3.72 g (62%). MS (m/e): 228, 226 (M⁺), 211, 209, 183, 181, 171, 169, 147, 118, 102, 91. ¹H-NMR (acetone-D₆): 4.29 (t, 1H, J=6 Hz) 4.73 (d, 2H, J=6 Hz) 7.32 (m, 2H) 7.51 (d, 1H, J=8 Hz) 7.78 (dd, 1H, J=8 Hz, 2 Hz). ¹³C-NMR (acetone-D₆): 54.74, 110.64, 119.96, 123.28, 124.57, 126.51, 128.12, 155.37.

C. [2-(2-formyl-5-furanyl)-3-hydroxymethyl benzofuran]. To the microwave vial was added the bromo-benzofuran derivative from B (0.1 mmol), the 2-formylfuran-5-boronic acid (Aldrich) (1.5 eq.), potassium carbonate (1.5 eq.), palladium dibenzylidene acetone (0.03 eq.) and degassed dimethylacetamide (1 ml). The mixture was blanketed with N₂ and heated in the microwave at 120° C. for 10 minutes (initial power 50 W). Water (2 ml) was added and the mixture was extracted with ether (4×) and the crude extract was adsorbed on silica gel and purified by MPLC (hexanes/ethyl acetate gradient). MS (m/e): 242 (M⁺), 225, 213, 196, 185, 168, 157, 139, 128, 102, 77. ¹H-NMR (acetone-D₆): 5.15 (s, 2H), 7.19 (d, 1H, J=4 Hz), 7.35 (dd, 1H, J=8 Hz, 1 Hz), 7.44 (dd, 1H, J=8 Hz, 1 Hz), 7.56-7.65 (m, 2H), 7.93 (d, 1H, J=8 Hz), 9.76 (s, 1H). ¹³C-NMR (acetone-D₆).

Radiolabeling of 49b: Preparation of [³H]2-bromo-3-hydroxymethyl benzofuran. To aldehyde 2-bromo-3-formyl-benzofuran (15 mg, 66 μmol) in propan-2-ol:water 4:1 (600 μl) was added a solution of NaBT₄ (5 Ci @ approx. 56 Ci/mmol) in propan-2-ol:water 4:1 (600 μl). The solution was then stirred at room temperature for 2 hours. The residue was dissolved in ethyl acetate (5 ml) and a sample was analyzed by silica TLC eluting in dichloromethane:methanol (95:5). Yield: 15 Ci/mmol, 260 mCi (17 μmol).

Preparation of [³H]2-Br-3-acetoxymethyl benzofuran. Three equivalents of acetic anhydride (5 μL) were added to 17 μmol of [³H]2-bromo-3-hydroxymethyl benzofuran, and the acetylation was monitored by TLC. After 2 hours, an additional 10 μl of acetic anhydride was added and the mixture was swirled and left overnight. After a total of 18 hours the reaction proceeded approximately 50%. A further 50 μl of acetic anhydride was added and the mixture was left for a further 2 hours. An additional 50 μl of acetic anhydride was added and the reaction mixture was left for a second night, after which the reaction appeared to have progressed to near completion. The crude mixture was purified by HPLC using an Ultrasphere (Beckman Coulter) ODS column eluting with a 0.1% TFA in water/acetonitrile gradient. The [³H]2-Br-3-acetoxymethyl benzofuran fractions were rotary evaporated to dryness.

Preparation of [³H]49b from [³H]2-Br-3-acetoxymethyl benzofuran. To [³H]2-Br-3-acetoxymethyl benzofuran (100 mCi) was added K₂CO₃ (1.4 mg), 5-formyl-furan-2-boronic acid (1.4 mg), Pd₂ dba₃ (0.2 mg), and degassed dimethylacetamide (400 μl). The mixture was blanketed under nitrogen gas and heated with stirring at 80° C. for 6 hours. The reaction mixture was analyzed by TLC silica eluting in CH₂Cl₂:MeOH (95:5). Deacetylation was performed by adding sodium hydroxide, 0.5 mg in THF:methanol (1:1), to the mixture. The reaction was swirled and stirred at room temperature. Samples were periodically analyzed by TLC and after 3 hours the reaction mixture was rotary evaporated to a lower volume and applied to a 2 g Sep-Pak cartridge. The required fraction was counted and analyzed and purified by HPLC using an Ultrasphere C18 column eluting with a water/methanol gradient, followed by another purification by HPLC using an Ultrasphere C18 column eluting with a water/acetonitrile gradient. The final product was analyzed by HPLC and mass spectrometry. Yield: specific activity of 13 Ci/mmol and 96.7% radiochemical purity.

### Example 4

### Preparation of the Compound of Formula III

The compound of formula III was synthesized according to Reaction Scheme 2 shown below.

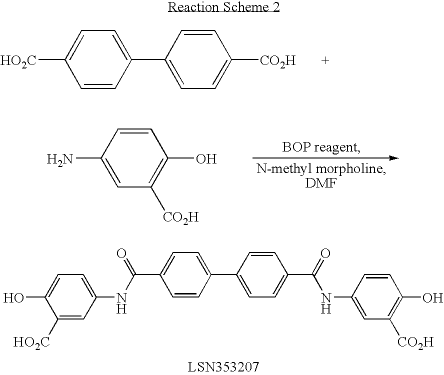

To a stirred slurry of the 4,4′-biphenyldicarboxylic acid (CAS #787-70-2, 978 mg, 4.038 mmol) in DMF (20 mL) under an atmosphere of nitrogen cooled in an ice bath to 0° C. was added the BOP reagent (CAS #56602-33-6, 3.661 g, 8.277 mmol) followed by the N-methyl morpholine (3.11 mL, 28.26 mmol). The resultant white mixture was stirred at 0° C. for 45 minutes, then the 5-amino salicylic acid (CAS#89-57-6, 1.268 g, 8.277 mmol) was added and the cooling bath removed. The mixture became a solution after 30 minutes of stirring and was stirred for 4.5 hours, while warming to ambient temperature. The resultant light brown solution was poured into 1 N aqueous HCl and a gray precipitate formed. The gray solid was collected by filtration, rinsing with water. Then, 1:1 MeOH/CHCl3 was added to the solid, which did not dissolve, and therefore the solid was filtered while rinsing with 1:1 MeOH/CHCl3. The collected gray solid (˜1.5 g) was suspended in DMF/water and heated over a steam bath without dissolving and therefore was filtered. The collected gray solid was dried in a vacuum oven at 60° C.-80° C. 1H-NMR (D6-DMSO, 400 MHz): 6.9d, J=2 Hz, 2H, 7.65-8.30m, 12H, 10.3s, 2H. FAB-MS: 513.6 (M+H+); elemental analysis: calculated: C, 65.62; H, 3.93; N, 5.47; found: C, 65.36; H, 4.21; N, 5.64.

The selectivity of the panel of binders was demonstrated by scintillation proximity assays and atomic force microscopy (AFM). SPA employs direct binding of a radiolabeled probe, and demonstrated both saturability and self-competition. For SPA and atomic force microscopy experiments, preparations of soluble oligomers and fibrils contained 20% biotinylated beta-amyloid 1-42 and 1-40, respectively (H-5642, H-5914, Bachem).

### Example 5

### Binding Assays

Lyophilized Streptavidin-Ysi beads (GE Healthcare) were reconstituted to 100 mg/mL in deionized water, and then further diluted in deionized water to give a suspension containing 0.25 mg/10 μL water. Unlabeled 49b, PIB and compound of formula III (353207) were dissolved in DMSO to give a final stock concentration of 15 mM.

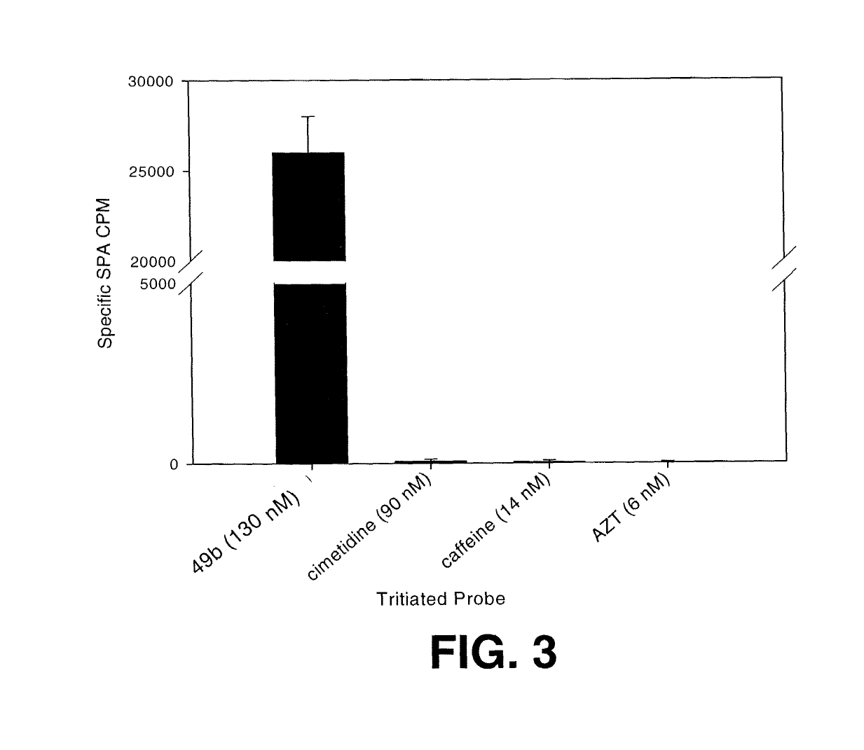

For the saturation-binding assay, isotopic dilution of radiolabeled 49b with unlabeled 49b was performed. ³H-49b was added to 500 uM of unlabeled 49b to a final concentration of 50 μCi/mL ³H-49b. The probe solution was then serially diluted in PBS to give six solutions giving 6.6, 13.1, 26.3, 52.5, 105, or 210 uM in the final assay well. Each concentration of the probe solution was incubated with 12 μg beta-amyloid 1-42 oligomer (20% biotin) or 1-40 (20% biotin) fibrils in a total volume of 90 μL. Assays were incubated at room temp for 2 hrs before addition of 10 μL YSi-streptavidin (0.25 mg) SPA bead. Assays were set-up in triplicate in 96-well NBS plates (Costar). Assays were incubated overnight and counted the following morning. Tritium-labeled 49b was compared with tritium-labeled cimetide, caffeine, and AZT in binding to soluble oligomers (FIG. 3). The specific activities of four probes were different, however the level of activity was the same at the concentrations where the probes were tested. For this assay, 130 nM of ³H-49b, 90 nM ³H-cimetidine, 14 nM ³H-caffeine, and 6 nM ³H-AZT were tested for direct binding to 12 ug of beta-amyloid-42 oligomer (20% biotin). Assay conditions were the same as described in saturation binding assay.

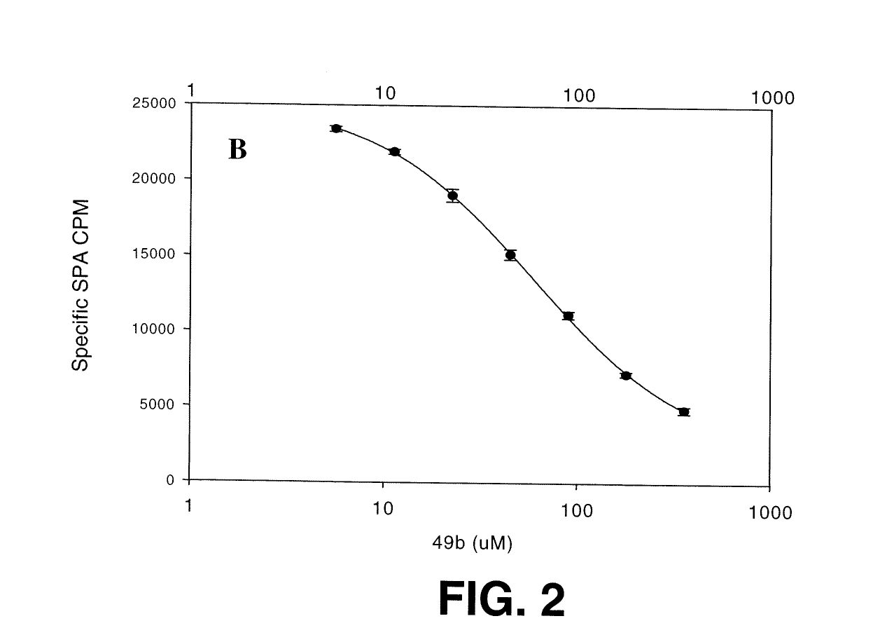

For the self-competition binding assay, assay wells contained 130 nM ³H-49b and the indicated amount of unlabeled 49b (FIG. 2). Assay conditions were the same as described in saturation binding assay.

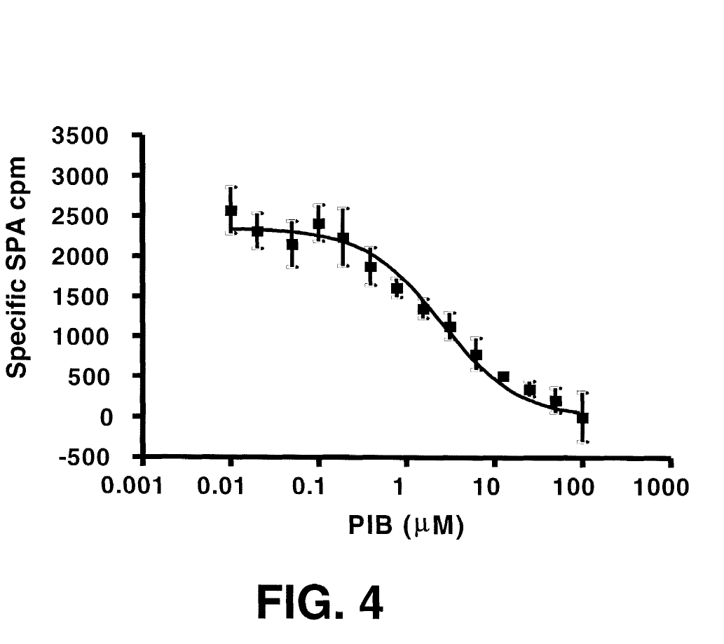

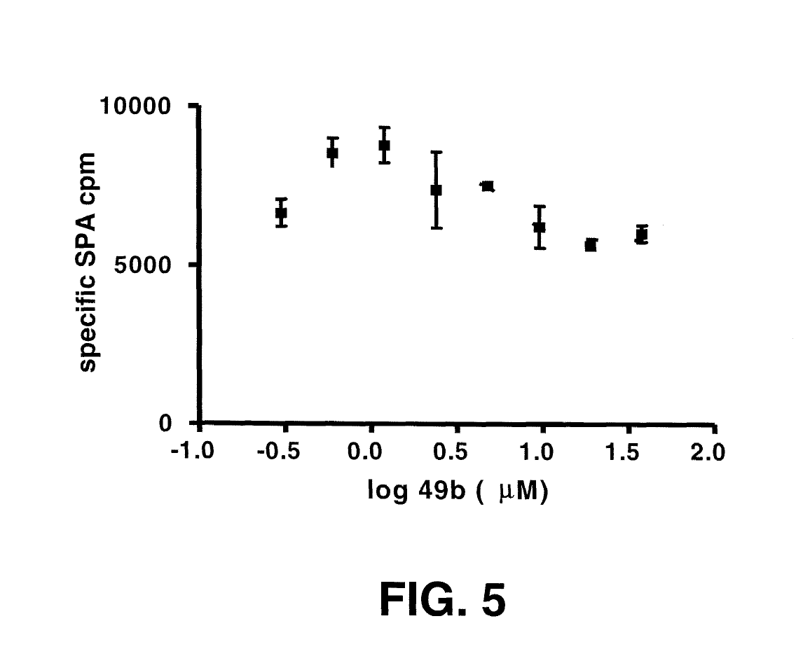

For the ³H-PIB binding reaction, 10 μg Aβ 1-40 fibrils were incubated with ˜30 nM [³H]PIB in the presence of the unlabeled competitors in a total volume of 90 μl PBS. Assays were set up in Corning Costar 96 well plates (3632) (FIGS. 4 and 5). Non-specific binding wells contained PBS instead of fibril/oligomer. Assays were incubated at room temperature for 2 hr before addition of 10 μl YSi-streptavidin bead (0.13 mg). Assays were shaken for 1 min and then incubated overnight before counting the following morning using Microbeta.

Data analyses were performed using Sigma Plot or GraphPad Prism® version 4. Curves were fitted using non-linear regression. K_{D} values and IC₅₀ values were estimated from the binding curves.

A direct binding assay using tritium labeled 49b was performed against oligomers or fibrils by scintillation proximity assay (SPA). Beta-amyloid containing 20% biotin and tritiated 49b were incubated in solution for two hours prior to addition of the Ysi-Streptavidin beads. While the binding reaction was done in solution, we wished to determine if beta-amyloid soluble oligomers and fibrils retained their respective conformation when bound to the SPA beads by AFM.

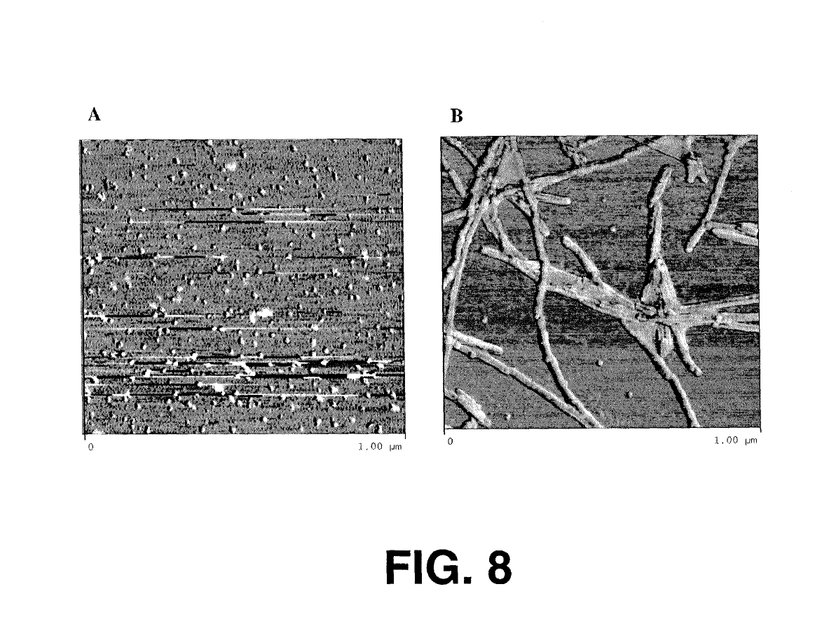

In the AFM studies, PVT-streptavidin SPA beads were used in place of Ysi-streptavidin beads because the smoother surface of the PVT beads enhanced visibility of the bound beta-amyloid. AFM on PVT-streptavidin beads demonstrated that the soluble oligomers and fibrils maintained distinct structure when bound to beads (FIG. 8).

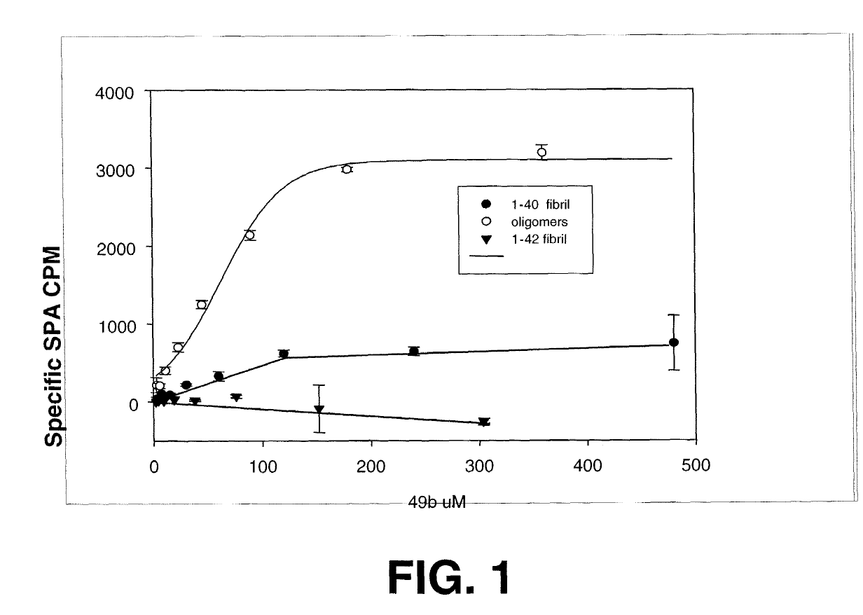

Saturation binding of 49b indicated a 50×10⁻⁶ mol/L binding constant when incubated with oligomers and a Bmax of 60-80 nmol/mg beta-amyloid, or a molar ratio of 1 probe:3-4 beta-amyloid). Much less binding was observed with 1-40 fibrils, and no measurable affinity could be determined when 49b was incubated with 1-42 fibrils under the same conditions used to measure binding to soluble oligomers (FIG. 1). Tritium labeled 49b binding to oligomers was competed by unlabeled 49b with an IC50 of 60 μM (FIG. 2). Tritium labeled non-benzofuran compounds (AZT, cimetidine, caffeine) did not bind to soluble oligomers indicating specificity of the benzofuran class (FIG. 3). This data demonstrated that 49b showed specificity to oligomers over fibrils.

A competition-binding assay using tritium labeled PIB was performed against fibrils (FIG. 4). PIB is a known binder to fibrillar A-beta and served as a positive control in this assay. Tritium labeled PIB binding to fibrils was competed by unlabeled PIB with an IC50 of 4 μM. Tritium labeled PIB binding to fibrils was not competed by unlabeled 49b. This demonstrated that PIB bound to A-beta-fibrils and 49b did not bind to fibrils.

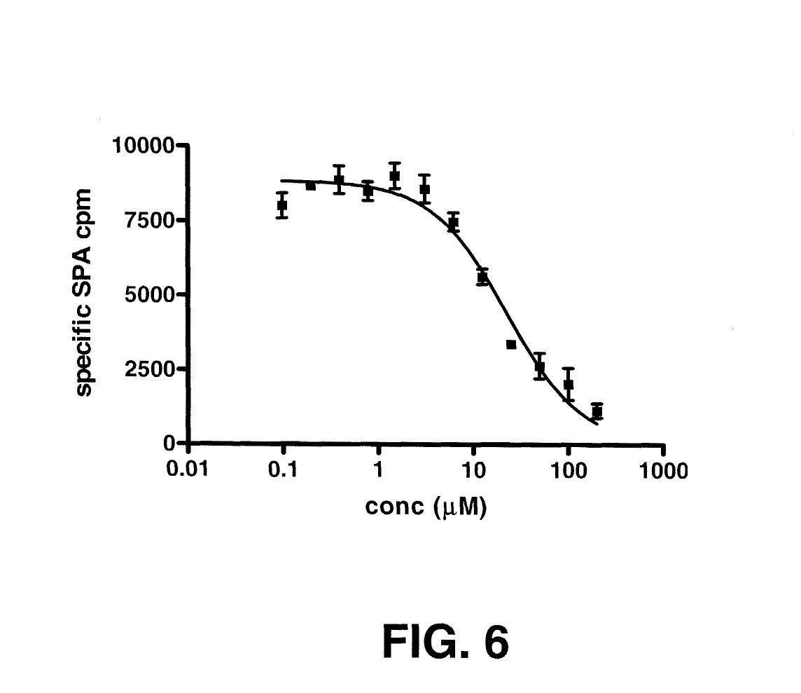

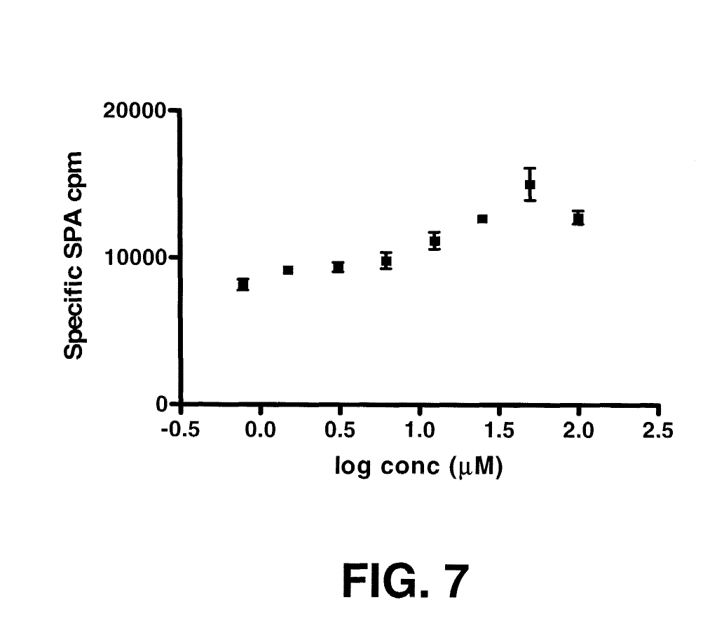

The compound of formula III competed with tritium-labeled 49b in binding to oligomers with an IC₅₀ between 1-7 μM (FIG. 6). This value was better than the IC₅₀ for unlabeled 49b, with an IC₅₀ of 60 μM. The compound of formula III (353207) did not compete for tritiated PIB binding to fibrils (FIG. 7).

### EQUIVALENTS

The invention may be embodied in other specific forms without departing from the spirit or essential characteristics thereof. The foregoing embodiments are therefore to be considered in all respects as illustrative rather than limiting on the invention described herein. The scope of the invention is thus indicated by the appended claims rather than by the foregoing description, and all changes that come within the meaning and range of equivalency of the claims are therefore intended to be embraced therein.

We claim:

1. A method of detecting soluble A-beta, comprising:
(a) providing a source of A-beta; and
(b) applying the compound of formula III to the source of A-beta 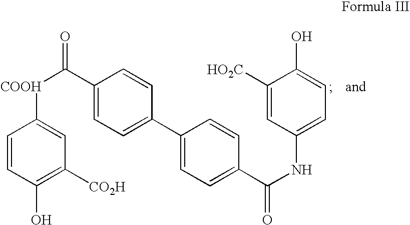
; and
(c) observing the binding of the compound of formula III to soluble A-beta present in the source of A-beta.

2. The method of claim 1, further comprising the step of conjugating the compound of formula III with a signal generator selected from a radioisotope, a paramagnetic particle, and an optical particle before applying to the source of A-beta.

3. The method of claim 1, further comprising the step quantifying the binding of compound of formula III to the soluble A-beta present in the source.

4. The method of claim 4, wherein the label comprises a radioisotope selected from ³H, ¹¹C, ¹⁴C, ¹⁸F, ³²P, ³⁵S, ¹²³I, ¹²⁵I, ¹³¹I, ⁵¹Cr, ³⁶Cl, ⁵⁷Co, ⁵⁹Fe, ⁷⁵Se, and ¹⁵²Eu.

5. The method of claim 4, wherein the label comprises a paramagnetic particle selected from ¹⁵⁷Gd, ⁵⁵Mn, ¹⁶²Dy, ⁵²Cr, and ⁵⁶Fe.

6. A method of determining the ability of a test agent to bind an A-beta species comprising the steps of:
(a) contacting a mammalian brain tissue sample with at least one A-beta species;
(b) applying a test agent to the brain tissue sample;
(c) determining whether the test agent binds to the A-beta species;
(d) repeating steps (a)-(c) using compound of formula III 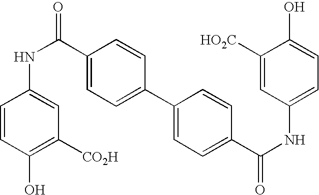
in place of the test agent.

7. The method of claim 6, further comprising the step of determining the relative binding of the test agent and the compound of formula III.

8. The method of claim 6, wherein, wherein each of steps (a)-(c) are repeated using a validated nonbinder.

9. The method of claim 6, wherein steps (a)-(c) performed in parallel or in tandem on both the test agent and the compound of formula III.

10. The method of claim 6, wherein the contacting step includes introducing the at least one A-beta species into an intact mammalian brain before the applying step.

11. The method of claim 6, wherein the amount of the A-beta species applied to the brain tissue sample is a concentration of about 0.1 pM to about 0.1 nM.

12. A composition comprising the compound of formula III

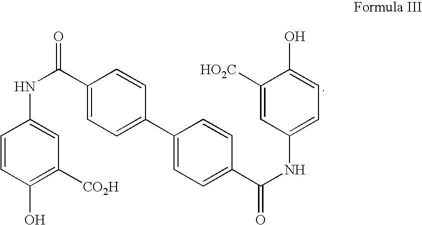

13. The composition of claim 12, wherein a signal generator selected from a radioisotope, a paramagnetic particle, and an optical agent is conjugated to the compound of formula III.

14. The composition of claim 13, wherein the signal generator comprises a radioisotope selected from ³H, ¹¹C, ¹⁴C, ¹⁸F, ³²P, ³⁵S, ¹²³I, ¹²⁵I, ¹³¹I, ⁵¹Cr, ³⁶Cl, ⁵⁷Co, ⁵⁹Fe, ⁷⁵Se, and ¹⁵²Eu.

15. The composition of claim 13, wherein the signal generator comprises a paramagnetic particle selected from ¹⁵⁷Gd, ⁵⁵Mn, ¹⁶²Dy, ⁵²Cr, and ⁵⁶Fe.
